SEQUENCE LISTING (10 sequences)

SEQ ID NO: 1 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 2 - protein, 916 aa, Homo sapiens
MDCEHSLSLPCRGAEAWEIGIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 3 - protein, 921 aa, Homo sapiens
MGQICQRESATAAEKPKLHLLAESEIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 4 - protein, 905 aa, Homo sapiens
MAKRALRDFIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 5 - protein, 917 aa, Homo sapiens
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 6 - protein, 25 aa, Homo sapiens
MIAAQLLAYYFTELKDDQVKKIDKY

SEQ ID NO: 7 - protein, 25 aa, Homo sapiens
MDCEHSLSLPCRGAEAWEIGIDKYL

SEQ ID NO: 8 - protein, 25 aa, Homo sapiens
MGQICQRESATAAEKPKLHLLAESE

SEQ ID NO: 9 - protein, 25 aa, Homo sapiens
MAKRALRDFIDKYLYAMRLSDETLI

SEQ ID NO: 10 - protein, 25 aa, Homo sapiens
MIASHLLAYFFTELNHDQVQKVDQY

(19) United States
(12) Patent Application Publication
(10) Pub. No.: US 2024/0299323 A1
(43) Pub. Date: Sep. 12, 2024

(54) CELLULAR ENERGY INHIBITOR FORMULATIONS FOR THE TREATMENT OF PATHOGENIC INFECTIONS AND ASSOCIATED METHODS

(71) Applicant: KoDiscovery, LLC, Baltimore, MD (US)

(72) Inventor: Young Hee Ko, Baltimore, MD (US)

(21) Appl. No.: 18/003,706

(22) PCT Filed: Jun. 29, 2021
(86) PCT No.: PCT/US2021/039722

§ 371 Date: Dec. 29, 2022

Related U.S. Application Data
(60) Provisional application No. 62/705,475, filed on Jun. 29, 2020.

(51) Int. Cl. A61K 31/19; A61K 45/06; A61K 47/12

(52) U.S. Cl. CPC A61K 31/19; A61K 45/06; A61K 47/12

(57) ABSTRACT

Compositions and methods for protecting a subject against, or treating a subject with, a pathogenic infection are presented. A method includes administering a composition including a cellular energy inhibitor having the structure according to formula I

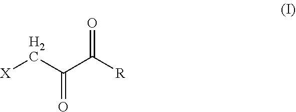

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal; where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl, at least one sugar to stabilize the cellular energy inhibitor and a biological buffer present in an amount sufficient to at least partially deacidify the cellular energy inhibitor and neutralize metabolic by-products of the cellular energy inhibitor.

### BACKGROUND

Infectious diseases are disorders caused by pathogens, such as bacteria, viruses, fungi, or parasites, to name a few. Some pathogens live in and on the human body, becoming infectious at times when the host subject's immune system is compromised. Other pathogens, however, encounter a subject by chance and infect through direct infiltration through the eyes, mouth, nose, etc. In some cases, chance encounters can be opportunities whereby the pathogen passed from one subject to another. In other cases, chance encounters may be animal or insect transmission, consumption of contaminated food or water that has been exposed to pathogens.

### SEQUENCE LISTING

This application contains a sequence listing which is incorporated herein by reference in ASCII format named 2553_026_PCT_US_Sequence_Listing.txt, created Aug. 18, 2023, and is 41 KB in size. The sequences contained in the sequence listing are found throughout the originally filed application.

p

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein but are merely representative thereof. One skilled in the relevant art will also recognize that the technology can be practiced without one or more of the specific details, or with other methods, components, compounds, ingredients, etc. In other instances, well-known materials, or operations may not be shown or described in detail to avoid obscuring aspects of the disclosure.

In this application, “comprises,” “comprising.” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including.” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The compositions of the present invention may include a pharmaceutically acceptable carrier and other ingredients as dictated by the particular needs of the specific dosage formulation. Such ingredients are well known to those skilled in the art. See for example, Gennaro, A. Remington: The Science and Practice of Pharmacy 19^{th} ed. (1995), which is incorporated by reference in its entirety.

As used herein, “administration,” and “administering” refer to the manner in which a composition is presented to a subject. Administration can be accomplished by various art-known routes such as enteral, parenteral, transdermal, and the like, including combinations thereof in some cases. Thus, an enteral administration can be achieved by drinking, swallowing, chewing, sucking of an oral dosage form comprising an active agent or other compound to be delivered. Parenteral administration can be achieved by injecting a drug composition intravenously, intra-arterially, intramuscularly, intrathecally, subcutaneously, etc. Transdermal administration can be accomplished by applying, pasting, rolling, attaching, pouring, pressing, rubbing, etc., of a transdermal preparation onto a skin surface. These and additional methods of administration are well-known in the art.

As used herein, “subject” refers to a mammal that may benefit from the administration of a drug composition or method of this invention. Examples of subjects include humans, and other animals such as horses, pigs, cattle, sheep, goats, dogs (felines), cats (canines), rabbits, rodents, primates, and aquatic mammals. In one embodiment, subject can refer to a human.

As used herein, “effective amount” or “therapeutically effective amount,” or similar terms, refers to a non-toxic but sufficient amount of a drug to achieve therapeutic results in treating a condition for which the drug is known to be effective or has been found to be effective as disclosed herein. Various biological factors may affect the ability of a delivered substance to perform its intended task or the amount of drug needed to provide a therapeutic result. Therefore, an “effective amount” or “therapeutically effective amount” may be dependent on such biological factors. The determination of an effective amount or therapeutically effective amount is well-within the ordinary skill in the art of pharmaceutical and medical sciences based on known techniques in the art as well as the present disclosure. See for example, Curtis L. Meinert & Susan Tonascia, Clinical Trials: Design. Conduct, and Analysis, Monographs in Epidemiology and Biostatistics, vol. 8 (1986).

As used herein, “drug.” “active agent.” “bioactive agent,” “pharmaceutically active agent,” “therapeutically active agent” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, or compounds significantly related thereto, including without limitation, prodrugs, active metabolites, isomers, and the like. The terms “cellular energy inhibitor,” “glycolysis inhibitor,” “mitochondrial inhibitor,” and the like, are considered to be active agents.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention but can be a referent to any minute incremental step of limiting or reducing a function through the full and absolute prevention of the function.

As used herein, “cellular energy inhibitor” refers to a compound that inhibits ATP production in a cell. In some examples, a cellular energy inhibitor can inhibit glycolysis, oxidative phosphorylation, or both glycolysis and oxidative phosphorylation in a cell.

As used herein, “glycolysis inhibitor” refers to a compound that inhibits, reduces, or stops, glycolysis in a cell.

As used herein, “mitochondria inhibitor” refers to a compound that inhibits, reduces, or stops mitochondrial production of ATP in a cell.

As used herein, the terms “dosage form,” “formulation,” and “composition” are used interchangeably and refer to a mixture of two or more compounds, elements, or molecules. In some examples, the terms “dosage form,” “formulation,” and “composition” may be used to refer to a mixture of one or more active agents with a carrier and/or other excipient.

As used herein, “carrier” or “pharmaceutically acceptable carrier” refers to a substance with which a drug may be combined to achieve a specific dosage formulation for delivery to a subject. In some examples, a carrier may or may not enhance drug delivery. As a general principle, carriers do not react with the drug in a manner that substantially degrades or otherwise adversely affects the drug, except that some carriers may react with a drug to prevent it from exerting a therapeutic effect until the drug is released from the carrier. Further, the carrier, or at least a portion thereof must be physiologically suitable for administration into a subject along with the drug.

As used herein, “admixed” means that at least two components of the composition can be partially or fully mixed, dispersed, suspended, dissolved, or emulsified in one another. In some cases, at least a portion of the drug may be admixed in at least one carrier substance.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art. For example, a data region that has an “increased” risk of corruption can refer to a region of a memory device which is more likely to have write errors to it than other regions in the same memory device. A number of factors can cause such increased risk, including location, fabrication process, number of program pulses applied to the region, etc.

As used herein, “cellular energy inhibitor” refers to a drug that inhibits, reduces, or stops ATP production in a cell. In some examples, a cellular energy inhibitor can inhibit glycolysis, oxidative phosphorylation, or both glycolysis and oxidative phosphorylation in a cell.

As used herein, “glycolysis inhibitor” refers to a drug that inhibits, reduces, or stops, glycolysis in a cell. In some examples, the cell can be an infected cell.

As used herein, “mitochondria inhibitor” refers to a drug that inhibits, reduces, or stops mitochondria function in a cell. In some examples, the cell can be an infected cell.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention but can be a referent to any minute incremental step of limiting or reducing a function through the full and absolute prevention of the function.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

The following technology provides various compounds, compositions, formulations, and the like, that can alleviate, treat, or otherwise protect against various pathogens and/or infectious conditions, including methods for alleviating, treating or protecting against such. Nonlimiting examples of such pathogens can include viruses, bacteria, parasites, and fungi. For the purposes of the present disclosure, prions can be considered to be pathogens. Furthermore, the present technology can reduce or eliminate at least a portion of a host immune response associated with a pathogenetic and/or infectious condition. As used herein, the term “infected cell” can be used to refer to any cell that has been infected with a pathogen. In some cases, the term “infected cell” can refer to an immune cell in a subject that, due to a pathogen infection, has been activated to a degree so as to cause undesirable effects in the subject. These “hyperactivated” immune cells can generate an excessive and uncontrolled immune response, often causing physiological damage beyond what is caused by the pathogen infection.

Various energy inhibitors can be utilized to treat a pathogen infection, to alleviate physiological symptoms caused by a pathogen infection, or to function as an adjuvant to protect a subject against acquiring a pathogen infection. In one example, the present technology can target the energy production of an infected cell. Without intending to be bound by any particular theory, certain cellular energy inhibitors can be used accomplish the aforementioned functions by such energy production targeting. While the energy metabolism reactions of eukaryotic cells are quite complex, there are two primary cellular energy production locations; the first is in the cytosol (glycolysis) and the second is in the mitochondria (oxidative phosphorylation). In the cytosol, sugar is split into pyruvate under aerobic conditions and lactate under anaerobic conditions. Under aerobic conditions, glycolysis converts one molecule of glucose into two molecules of pyruvate (pyruvic acid), generating energy in the form of adenosine triphosphate (ATP), a molecule that provides energy to the cell. In uninfected, normal cells, a small proportion of the total ATP production is derived from glycolysis, with a significant majority of ATP being produced via oxidative phosphorylation in the mitochondria. In infected cells, on the other hand, energy production in the cytosol via glycolysis can be significantly increased, which can result in a significant increase in lactic acid production. Many pathogens alter the energy metabolism of the cell to significantly increase glycolysis, even in the presence of oxygen, thus resulting in greatly increased lactic acid production. These cells begin to pump the lactic acid out via monocarboxylate transporters, which are greatly increased in infected cells compared to noninfected cells.

### Energy Inhibitors

One group of energy inhibitors that can be used in accordance with the present disclosure includes halopyruvate molecules. Such molecules can function to inhibit cellular energy production in infected cells, thus limiting the abilities of such infected cells to generate ATP. One nonlimiting example of a halopyruvate that is a useful cellular energy inhibitor is 3-bromopyruvate (3-BP). It is noted that, while 3-BP is used herein as an example molecule, other halopyruvate molecules should not be seen as limiting. 3-BP is a small molecule that has sufficiently similar chemical structure to lactic acid that it can enter infected cells through the upregulated lactic acid transport system. In some cases, 3BP can have little effect on normal cells as such cells contain very few lactic acid transporters in the uninfected state. Once in an infected cell, 3-BP damages glycolysis and oxidative phosphorylation due to its highly reactive nature, thus significantly reducing ATP production. This reduction in ATP production subsequently leads to the death of the infected cell. It is further noted that other cellular energy inhibitors not classified as halopyruvates but having a sufficiently similar chemical structure to enter infected cells via the lactic acid transporters, are also contemplated.

Additionally, the damage done to glycolysis and oxidative phosphorylation systems in infected cells by 3-BP damages described above can additionally limit or eliminate hyperactivated immune system cells that may cause sepsis via the same or similar mechanisms. During such infections, for example, white blood cells generally become activated and greatly increase their ATP production similar to infected cells. In cases where the white blood cells become hyperactivated (i.e., they begin to damage uninfected tissue in the subject) and normal tissue/cell damage occur, the resulting sepsis can be more damaging to the subject than the pathogen infection itself. By killing these hyperactivated immune cells, 3-BP can reduce further the damaging systemic effects that can occur as a result of such an infection.

As described above, pathogenic infection for many pathogens generally proceeds according to several stages, including 1) pathogen entry into a cell, 2) replication of the pathogen in the cell, 3) spread of the pathogen through the subject (i.e., host), and 4) release of the pathogen into the environment where other subjects can be infected. In various examples, a 3-BP compound can be administered in a manner appropriate for the stage of a pathogenic infection in a subject, which can include protecting the subject from being infected. It should therefore be understood that the term “pathogenic infection” can also include prophylactic use of an adjuvant to provide protection for subjects not currently infected. In one example, 3-BP can be formulated into a dosage form appropriate for an administration route capable of treating the stage of pathogenic infection.

In one example, 3-BP can be administered as an adjuvant to protect a subject from acquiring a pathogenic infection. Such protection can occur prior to pathogen entry into cells, after pathogen entry into cells, or both. In the case of protecting a subject from pathogenic infection prior to pathogen entry, 3-BP can disturb or otherwise disrupt receptor binding proteins that pathogens utilize to enter cells. For such cases, 3-BP can be delivered to cellular surfaces to affect such disruption of receptor binding proteins. Any dosage form capable of being delivered to cellular surfaces is contemplated, nonlimiting examples of which can include sprays, aerosols, powders, liquids, ointments, creams, wipes, and the like, including combinations thereof. In one example of prophylactic use of 3-BP, the throat, mouth, lungs, nose, and/or the like can be coated to provide protection against such infection. For example, a subject can be protected against infection by delivering aerosols, sprays, powders, or the like into the mouth, nose, lungs, throat, etc. Gargling with a liquid formulation can also provide protection from infection to the mouth, throat, and any other cellular surface contacted by the gargling action. In some cases, a nebulizer can be used to administer a liquid/vapor 3-BP formulation to the lungs. In other cases, liquid drops can be used to deliver 3-BP to the eye of a subject. Thus, by disrupting the cell surface proteins used by the pathogen to enter, the cell is protected from infection.

In some examples, prophylactic protection can still be accomplished after pathogen entry into cells. As described above, most viral pathogens insert genetic material into the cell cytosol, where replication/activation of the genetic material allows the pathogen to take control of the cell's genetic machinery. 3-BP can break up genetic material, such as RNA and DNA, present in the cytosol, thus inactivating the pathogen genome structure and preventing pathogenic takeover of the cell. As with the above, the dosage form of the 3-BP formulation can be appropriate for the location of the infection, which understanding is well within the knowledge of those skilled in the art.

Furthermore, as has been described above, once the cells have been infected, they significantly increase their ATP production through glycolysis and oxidative phosphorylation. In this case, 3-BP can enter the cell through the monocarboxylate transport process to disrupt both ATP production processes. As with the above, the dosage form of the 3-BP formulation can be appropriate for the location of the infection, which understanding is well within the knowledge of those skilled in the art.

For many pathogenic infections, the resulting cellular death causes a so called “cytokine storm,” which can cause increased further damage to a subject. Thus, through prophylactic protection, limiting the increase of ATP production in infected cells, gradually killing infected cells and activated immune cell, and the like, 3-BP formulations can function to reduce viral load and limit the cytokine storm.

It is noted that death of a cell can occur when ATP is being used at a higher rate than it is being produced. The death of a subject can occur when sufficient numbers of key cells die due to the lack of sufficient ATP production. Infections that limit oxygen to a patient, such a virus or the septic effects of a virus that attack lung tissue, for example, can greatly decrease ATP production, not only in the affected tissue, but system wide. In one example, therefore, the present compositions can additionally include ATP to allow a subject to overcome such ATP-limiting effects of such infections.

The present disclosure provides a 3-BP composition that can be given to treat pathogenic infections or as a adjuvant to protect against a pathogenic infection, referred to hereinafter as a Glycolytic/Glyoxylate Inhibitor (“GGIs”). GGI can treat or reduce the effects of a pathogenic infection, as well as acting as an adjuvant to protect against such infection. Without intending to be bound by any scientific theory, GGI can function in a variety of ways to combat a pathogenic infection. For example, GGI can cause a depletion or reduction of ATP from both cellular energy production pathways, namely glycolysis and oxidative phosphorylation. By reducing or depleting ATP production, the pathogen is either directly inactivated or, in the case of viral infection, for example, the infected cell in which the virus is reproducing is eliminated. In another example, GGI can cause a disruption of the glyoxylate cycle, which is an anabolic pathway occurring in plants, bacteria, protists, and fungi. GGI can inactivate the microbial enzymes isocitrate lyase and malate synthase, which are essential to pathogen survival when grown in nutrient-deficient microenvironments. By disrupting this pathway, GGI eliminates the ability of a pathogen from switching to the glyoxylate cycle.

### Primary Infections

In some cases, a pathogen infection can be a primary infection, and as such, an energy inhibitor composition can be used in the treatment of such infections. In one example, a primary infection can be an initial infection of a subject by a pathogen that is the root cause of the subject's current infection. In another example, a primary infection can be categorized in terms of the status of a subject's immune system. For example, a primary infection can be described as an infection caused by the activity of a pathogen within a normal, healthy subject. The ability of the pathogen to infect and spread through such a healthy subject is dependent on the intrinsic virulence of the pathogen and the level of protection afforded by the subject's immune system.

As a general example of a primary infection, a virus initially needs to enter a cell in order to reproduce and establish a viral infection. The virus enters the cell through interaction with cell surface proteins that allow the virus to attach to the cellular membrane of the cell. Following attachment, a hole is formed in the cell membrane through which genetic material from the virus enters. Depending on the type of virus, the genetic material can be RNA or DNA. Once inside the cell, the viral genetic material takes control of the cell's genetic machinery and the cell generally begins to replicate the virus, which is facilitated by an increase in the cellular processes for producing ATP. Newly created viruses can exit the cell via various mechanisms, including gradual release through budding of the cell membrane or rupture of the cell. Regardless of the mechanism, such release thus allows the virus to spread further to infect more of the subject's tissue, as well as exiting the subject to spread the infection to other subjects. Given the dependence of viral replication on ATP, a viral infection (or other pathogenic infection) can be treated by attacking the ATP production mechanisms of infected cells.

### Secondary Infections

In some cases, a pathogen infection can be a secondary infection, and in such cases, an energy inhibitor composition can often be used in the treatment of such infections. In one example, a secondary infection can be a complication or further pathogenic infection following a primary infection. In other words, a secondary infection can include any pathogenic infection following, or coinfecting with, a primary infection. Secondary infections can include superinfections, coinfections, opportunistic pathogen infections, and the like.

A superinfection, for example, is a process whereby a subject's cells that have previously been infected by a virus become co-infected, at a later point in time, by a different strain of the virus, a different virus, or the like. In some cases, viral superinfections may be resistant to the antiviral drug that was used to treat the original viral infection. Viral superinfections can also be less susceptible to the subject's immune response compared to the initial superinfection.

In another example, an opportunistic pathogen can cause a secondary infection in subjects having depressed or otherwise compromised immune systems or in abnormal openings into a subject's body. In some cases, opportunistic infections can be caused by pathogens that are ordinarily in contact with the subject, but that are unable to cause infection due to the subject's immune system. Once the immune system becomes compromised, such pathogens are able to infect the subject. In some examples, opportunistic bacterial infections can opportunistically infect a subject following a viral infection that has lowered the subject's immune system. In other examples, a subject's immune system can be depressed as a result of a genetic condition, immunosuppressive drugs, such as cancer therapy drugs, or through any condition that negatively affects the immune system.

Various concurrent coinfections and superinfections can greatly increase mortality rates as compared to the primary pathogenic infection alone. In such cases, an energy inhibitor can be given to a subject in order to combat such secondary infections to effectively lower the mortality rates associated with the primary infection. Additionally, an energy inhibitor can be given as an adjuvant to the primary infection, thus providing protection to the subject. Additionally, an energy inhibitor can be given as an adjuvant to a secondary infection. In other words, a subject infected with the primary pathogen infection can be protected against acquiring a secondary infection through adjuvant therapy using an energy inhibitor.

Under conditions of viral and other pathogenic infection the immune system response may become overly aggressive. For example, neutrophils, macrophages and dendritic cells can become hyperactivated in fighting the pathogen infection, leading to an increase in glycolysis in these immune system cells. One result from the hyperactivated cells is a large increase in cytokine production. It has been observed that interleukin-6 (IL-6) is significantly elevated in some pathogen infections, as is the case in many COVID-19 infections caused by the SARS-COV-2 virus. IL-6 is a predominant cytokine in the COVID-19 infection and IL-6 levels appear to correlate with COVID-19 disease severity. IL-6 appears to be driven into overexpression by hyperglycoloysis as a result of the hyperactivation of at least neutrophils, macrophages and dendritic cells. These and other hyperactivated immune cells cause further increases in cytokine production, eventually generating an inflammatory cytokine amplification loop, known as hypercytokinemia or the “cytokine storm.”

GGI (3-BP composition) of the present disclosure can be therapeutically administered to an individual having a pathogen infection who is experiencing, is at risk for developing, or has a need to be protected from, IL-6 overexpression leading to hypercytokinemia. GGI has the effect of reducing cytokine production due to GGI's effect on glycolysis and oxidative phosphorylation, stopping the cytokine amplification loop, and reducing or eliminating, including protecting from, they cytokine storm. This glycolytic normalization as a result of GGI administration additionally leads to reduced pro-thrombotic conditions and an improvement in vascular integrity.

Additionally, GGI administration can reduce or eliminate the SARS-COV-2 hyperactivation of endothelial and pericyte cells that can be related to vascular dysfunction. For example, a hyperactivated inflammatory attack on endothelial cells EC, as well as any existing comorbidities, such as diabetes, hypertension, heart disease, obesity, and the like, increase endothelial cell permeability and weaken close junctures with pericytes, the support cells that surround endothelial cells to support normal circulation of capillaries and veins. These cells have a high expression of angiotensin converting enzyme-2 (ACE2) receptors when infected (e.g., by SARS-COV-2). This condition allows for virus pass-through to reach and attach the Pericytes, with exacerbation of microvascular dysfunctionality and increasing potential for pulmonary fibrosis, Blood Brain Barrier penetration (via infected Pericytes), and other thrombotic response. GGI (3-BP composition) of the present disclosure can be therapeutically administered to an individual having a pathogen infection to “normalize” glycolysis to reduce pro-thrombotic conditions and improve vascular integrity.

IL-6 induces cellular factors that are known to be problematic vis-à-vis COVID-19 disease progression and severity. IL-6 induces Vascular Endothelial Growth Factor (VEGF), which in turn drives abnormal angiogenesis in the lungs of subjects experiencing COVID-19 infections, which can be about 28 times higher than in the lungs of unaffected subjects.

In the early process of this neovascularization, fibrous exudate is produced in the nascent blood capillaries, which drives up localized fibrin deposition and related D-dimer levels. IL-6 also induces Plasminogen Activator Inhibitor-1 (PAI-1), which in turn counters fibrinolysis, thus hindering the process of dissolving vascular micro-clots and fostering what has been called “fibrinolysis shutdown.” This effectively increases the potential for deadly thrombotic events. As another effect, IL-6 stimulates platelet hyperactivity, which leads to excessive release of pro-coagulatory factors with implications for thrombi formation. Furthermore, activated platelets have been shown to suppress pulmonary fibrinolysis, which leads to coagulatory dysfunction and potentially increased mortality.

One key agent blocking fibrinolysis (blood clot dissolution) is PAI-1 which in COVID-19 is highly elevated. Activated platelets, heightened IL-6, and increased VEGF production—all typical of severe COVID-19 infections—are key drivers of elevated PAI-1 expression. GGI administration can greatly reduce PAI-1's IL-6 levels that were elevated in response to a pathogen infection, such as COVID-10, for example, thus mitigating PAI-1's negative effect on fibrinolysis (aka “Fibrinolysis Shutdown”), leading to a decrease in mortality rates in affected individuals.

In another example, as endothelium becomes inflamed and sub-endothelial matrix tissue is exposed, adhesion molecules are expressed. Such expression triggers platelets to heal the damaged tissue. However, excessive inflammatory cytokines, collagen interactions, and antibody release drive platelets into a state of hyperactivation, accompanied with shift to a heightened “glycolytic phenotype.” Subsequently, platelets and other clotting molecules can become implicated in microthrombi, venous thromboembolism, and myocardial events-leading to increased mortality. GGI administration to an infected subject can function to moderate hyperglycolysis and other confirmed inhibition targets, such as tyrosine phosphatase and pyruvate dehydrogenase complex, for example. Such moderating influence can thereby effectively reduce expression of platelet activation markers, diminish platelet reactivity, and abrogate platelet aggregation.

In accordance with this, the present disclosure provides various cellular energy inhibitors to alleviate, treat, or otherwise protect against various pathogens and/or infectious conditions, including methods for alleviating, treating or protecting against pathogenic infections. One nonlimiting example of such a cellular energy inhibitor is shown according to formula I.

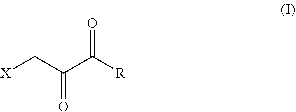

Various molecules are contemplated, wherein, for example, X can be, without limitation, a nitro, an imidazole, a halide, sulfonate, a carboxylate, an alkoxide, amine oxide, or the like. Additionally, R can be, without limitation, OR′, N(R″)₂, C(O)R′″, C1-C6 alkyl, C6-C12 aryl, C1-C6 heteroalkyl, a C6-C12 heteroaryl, H, an alkali metal or the like, where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl.

Additionally, the cellular energy inhibitor composition can include a variety of excipients, active agents, prodrugs, metabolites, buffers, and the like, such as, for example, one or more sugars, polyalcohols, or the like, glycolysis inhibitors, biological buffers, and the like. In some examples the cellular energy inhibitor molecule can be formulated in a composition with at least one sugar, which can stabilize the cellular energy inhibitor by substantially preventing the inhibitor from hydrolyzing.

In one example, R of formula (I) can be OH and X of formula (I) can be a nitro, an imidazole, a halide, a sulfonate, a carboxylate, an alkoxide, an amine oxide, or the like. Additionally, X can be a halide, such as, for example, fluoride, bromide, chloride, iodide, or the like. In one example, X can be a sulfonate, such as, for example, a triflate, a mesylate, a tosylate, or the like. In another example, X can be amine oxide. In still another example, the amine oxide can be dimethylamine oxide.

In one example, the cellular energy inhibitor can be a 3-halopyruvate, such as, for example, 3-fluoropyruvate, 3-chloropyruvate, 3-bromopyruvate, 3-iodopyruvate, or a combination thereof. A general structure showing a halide in the 3-position is shown in formula II.

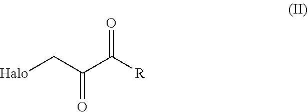

In a further nonlimiting example, the cellular energy inhibitor can have bromine in the 3-position, as shown in formula III.

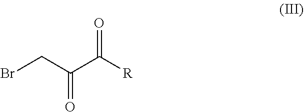

In one further nonlimiting example, the cellular energy inhibitor can be 3-bromopyruvate (3-BP), as shown in formula IV.

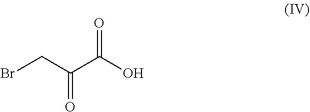

As such, in one example, the cellular energy inhibitor in the cellular energy inhibitor composition can be 3-BP (i.e., a 3-BP composition). It is noted that, while 3-BP used herein as an example molecule in describing a cellular energy inhibitor and a cellular energy inhibitor composition, such should not be seen as limiting.

In some examples, a composition can include 3-BP an at least one sugar, at least two sugars, at least three sugars, and the like. In one example, a sugar can include a monosaccharide, a disaccharide, an oligosaccharide, or a combination thereof. Nonlimiting examples of monosaccharides can include glucose, fructose, galactose, and the like. Nonlimiting examples of disaccharides can sucrose, lactose, maltose, and the like. It is noted that, for the purposes of the present disclosure, the term “sugar” can also include oligosaccharides, polysaccharides, polyols, and similar molecules that function to stabilize 3-BP.

A sugar can include a 3-carbon sugar, a 4-carbon sugar, a 5-carbon sugar, a 6-carbon sugar, a 7-carbon sugar, and the like, including combinations thereof. In one aspect, the sugar can be a 3-carbon sugar, a 4-carbon sugar, a 5-carbon sugar, a 6 carbon sugar, a 7-carbon sugar, and the like, including combinations thereof, provided the sugar is not involved in energy metabolism to the extent that it generates energy (i.e., a nonmetabolizable sugar).

Furthermore, in some examples the cellular energy inhibitors molecule can be formulated in a composition with at least one sugar, which can stabilize the cellular energy inhibitor by substantially preventing the inhibitor from hydrolyzing.

In one example, the sugar can be gluconic acid. In another embodiment, the sugar can be glucuronic acid. At least one of the sugars can be a five-carbon sugar. In one embodiment, at least two of the sugars can be five-carbon sugars. The five-carbon sugars can be independently selected from mannitol, erythritol, isomalt, lactitol, maltitol, sorbitol, xylitol, dulcitol, ribitol, inositol, or the like, including combinations thereof. In one example, at least one of the sugars can be glycerol. In another example, the sugars can be glycerol, inositol, and sorbitol. Other nonlimiting example of sugars can include ethylene glycol, threitol, arabitol, galactitol, fucitol, iditol, volemitol, maltotriitol, maltotetraitol, and polyglycitol, including combinations thereof. In one example, the sugars can include glycerol, inositol, sorbitol, mannitol or any combination thereof. In another example, the sugars can include glycerol, inositol, sorbitol, or any combination thereof. In other examples, the sugar can be a polyalcohol.

In some examples, the composition can include glycerol in a range from about 0.1 wt % to about 5.0 wt % or from about 0.1 wt % to about 3.0 wt %. In other examples, the composition can include inositol in a range from about 0.1 wt % to about 10 wt %, from about 0.1 wt % to about 5 wt %, or from about 0.5 wt % to about 1 wt %. In further examples, the composition can include sorbitol in a range from about 0.1 wt % to about 30 wt % or from about 0.1 wt % to about 20 wt %. In yet further examples, the composition can include mannitol in a range from about 0.1 wt % to about 30 wt % or from about 0.1 wt % to about 10 wt %. Additionally, each of the sugars may be added in a volume up to a maximum solubility of the sugar in the formulation or composition.

The sugars described herein can be any isomeric form. In one embodiment, the compositions described herein can include the less biologically active form of the sugar as compared to its isomer. In one aspect, the less biologically active sugar can be the L-enantiomer sugar. However, if the D-enantiomer sugar is found to be less biologically active as compared to its L form, then the D form can be used. In one embodiment, such sugars can function as a glycolytic inhibitor.

As discussed herein, the cellular energy inhibitor is taken up by an infected cell and metabolized, which results in certain metabolite by-products. In one embodiment, a by-product can be a hydrogen halide. Additionally, the hydrogen halide can be hydrogen bromide or hydrogen iodide. In one embodiment, the hydrogen halide can be hydrogen bromide.

Generally, 3-BP can be formulated as any type of dosage form capable of being delivered to a subject. Such dosage forms can be enteral, parenteral, transdermal, or the like. Enteral dosage forms can be sustained release or immediate release and can include, without limitation, tablets, lozenges, capsules, caplets, encapsulated pellets, encapsulated granules, encapsulated powders, gelatin capsules, liquids, syrups, elixirs, suspensions, sprays, aerosols, powders, and the like, including combinations thereof. Nonlimiting examples of transdermal dosage forms can include lotions, gels, creams, pastes, ointments, liquid sprays, liquid drops, powder sprays, wipes, emulsions, aerosols, transmucosal tablets, adhesive devices, adhesive matrix-type transdermal patches, liquid reservoir transdermal patches, microneedle devices, magnetic devices, and the like. Nonlimiting examples of parenteral dosage forms can include intravenous, subcutaneous, and the like.

The cellular energy inhibitor composition can additionally include a glycolysis inhibitor. Many suitable glycolysis inhibitors are contemplated, however a nonlimiting list can include 2-deoxy glucose (2-DG), lonidamine, imatinib, oxythiamine, 6-aminonicotinamide, genistein, 5-thioglucose (5-TG), mannoheptulose, α-chlorohydrin, omidazole, oxalate, glufosfamide, and the like, including combinations thereof. The cellular energy inhibitor composition can further include a hexokinase inhibitor.

In some examples, a 3-BP composition can include a biological buffer that is present in an amount sufficient to at least partially deacidify the cellular energy inhibitor and neutralize metabolic by-products of the cellular energy inhibitor. Nonlimiting examples of biological buffers can include a citrate buffer, a phosphate buffer, an acetate buffer, and the like, including combinations thereof. In one specific example, the biological buffer can be a citrate buffer. In yet another specific example, the biological buffer can be sodium citrate.

In some examples, the composition can comprise the biological buffer in a concentration of from about 0.1 mM to about 200 mM. In one embodiment, the composition can comprise the biological buffer in a concentration of from about 1 mM to about 20 mM. Additionally, the biological buffer can maintain a physiological pH of 4.0 to 8.5. In one embodiment, the biological buffer can maintain a physiological pH of 5.5 to 8.0. In another embodiment, the biological buffer can maintain a physiological pH of 6.8 to 7.8. In still another embodiment, the biological buffer can maintain a physiological pH of 7.3 to 7.6.

In addition to the above components, the 3-BP compositions described herein can further comprise a halo monocarboxylate compound that is separate from the cellular energy inhibitor. In cases where the halo monocarboxylate compound can function to inhibit glycolysis and/or mitochondria function, the halo monocarboxylate can be considered a second cellular energy inhibitor. In one embodiment, the halo monocarboxylate compound can be a halo two-carbon monocarboxylate compound. The halo two-carbon monocarboxylate compound can be selected from, without limitation, 2-fluoroacetate, 2-chloroacetate, 2-bromoacetate, 2-iodoacetate, and the like, including combinations thereof. In one embodiment, the halo two-carbon monocarboxylate compound can be 2-bromoacetate. In one example, the composition can comprise the halo two-carbon monocarboxylate compound in a concentration from about 0.01 mM to about 5.0 mM. In another example, the composition can comprise a halo two-carbon monocarboxylate compound in a concentration from about 0.1 mM to about 0.5 mM.

Additionally, a halo monocarboxylate compound can be a halo three-carbon monocarboxylate compound. In one embodiment, the halo three-carbon monocarboxylate compound can be selected from, without limitation, 3-fluorolactate, 3-chlorolactate, 3-bromolactate, 3-iodolactate, and the like, including combinations thereof. In another example, the composition can include the halo three-carbon monocarboxylate compound in a concentration from about 0.5 mM to about 250 mM. In one embodiment, the composition can comprise the halo three-carbon monocarboxylate compound in a concentration from about 10 mM to about 50 mM.

In some examples, the present 3-BP compositions described herein can further comprise an antifungal agent and/or antibacterial agent. In one embodiment, the composition can individually comprise the antifungal agent and/or antibacterial agent in a concentration from about 0.01 mM to about 5.0 mM. In another embodiment, the composition can individually comprise the antifungal agent and/or antibacterial agent in a concentration from about 0.05 mM to about 0.5 mM.

In some examples, the 3-BP compositions described herein can further comprise a mitochondrial inhibitor in addition to the cellular energy inhibitor. The mitochondrial inhibitor can be selected from, without limitation, oligomycin, efrapeptin, aurovertin, and the like, including combinations thereof. In another example, the composition can include the mitochondrial inhibitor in a concentration from about 0.001 mM to about 5.0 mM. In one example, the composition can include the mitochondrial inhibitor in a concentration from about 0.01 mM to about 0.5 mM.

In addition to the above concentrations, the present compositions can have various ratios of the components described herein. In one embodiment, the cellular energy inhibitor and biological buffer can be present in a ratio ranging from 1:1 to 1:5 by mM. In another embodiment, the cellular energy inhibitor and glycolysis inhibitor can be present in a ratio ranging from 5:1 to 1:1 by mM. In still another embodiment, the cellular energy inhibitor and the at least one sugar are present in a ratio ranging from 1:1 to 1:5 by mM. In yet another embodiment, the cellular energy inhibitor and the halo two-carbon monocarboxy late compound can be present in a ratio ranging from 20:1 to 4:1 by mM. In still yet another embodiment, the cellular energy inhibitor to mitochondrial inhibitor can be present in a ratio ranging from 20:1 to 40:1 by mM.

As described above, the present 3-BP compositions can comprise antifungal agents, antibiotics, glycolysis inhibitors, inhibitors of mitochondria, sugars, and biological buffers, without limitation. Examples of such agents include, but are not limited to, amphotericin B, efrapeptin, doxorubicin, 2-deoxyglucose (2DOG), d-lactic acid, analogs of 2DOG, dicholoracetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. Various active agents of the composition can include a cellular energy inhibitor, a glycolysis inhibitor, a mitochondria inhibitor, a halo monocarboxylate compound, an antifungal agent, an antibiotic agent, and the like.

As used herein, “hexokinase 1” or “hexokinase 1 isozyme” refers to any isoforms of hexokinase 1 and its naturally known variants, including those provided in SEQ ID NO: 1, SEQ ID NO: 2, SEQ ID NO: 3, and SEQ ID NO: 4, as follows:

| (SEQ ID NO: 1) | |
|---|---|
| 1 | MIAAQLLAYY FTELKDDQVK KIDKYLYAMR LSDETLIDIM TRFRKEMKNG LSRDFNPTAT |
| 61 | VKMLPTFVRS IPDGSEKGDF IALDLGGSSF RILRVQVNHE KNQNVHMESE VYDTPENIVH |
| 121 | GSGSQLFDHV AECLGDEMEK RKIKDKKLPV GFTFSFPCQQ SKIDEAILIT WTKRFKASGV |
| 181 | EGADVVKLLN KAIKKRGDYD ANIVAVVNDT VGTMMTCGYD DQHCEVGLII GTGTNACYME |
| 241 | ELRHIDLVEG DEGRMCINTE WGAFGDDGSL EDIRTEFDRE IDRGSLNPGK QLFEKMVSGM |
| 301 | YLGELVRLIL VKMAKEGLLF EGRITPELLT RGKFNTSDVS AIEKNKEGLH NAKEILTRLG |
| 361 | VEPSDDDCVS VQHVCTIVSF RSANLVAATL GAILNRLRDN KGTPRLRTTV GVDGSLYKTH |
| 421 | PQYSRRFHKT LRRLVPDSDV RFLLSESGSG KGAAMVTAVA YRLAEQHRQI EETLAHFHLT |
| 481 | KDMLLEVKKR MRAEMELGLR KQTHNNAVVK MLPSFVRRTP DGTENGDFLA LDLGGTNFRV |
| 541 | LLVKIRSGKK RTVEMHNKIY AIPIEIMQGT GEELFDHIVS CISDFLDYMG IKGPRMPLGF |
| 601 | TFSFPCQQTS LDAGILITWT KGFKATDCVG HDVVTLLRDA IKRREEFDLD VVAVVNDTVG |
| 661 | TMMTCAYEEP TCEVGLIVGT GSNACYMEEM KNVEMVEGDQ GQMCINMEWG AFGDNGCLDD |
| 721 | IRTHYDRLVD EYSLNAGKQR YEKMISGMYL GEIVRNILID FTKKGFLFRG QISETLKTRG |
| 781 | IFETKELSQI ESDRLALLQV RAILQQLGLN STCDDSILVK TVCGVVSRRA AQLCGAGMAA |
| 841 | VVDKIRENRG LDRLNVTVGV DGTLYKLHPH FSRIMHQTVK ELSPKCNVSF LLSEDGSGKG |
| 901 | AALITAVGVR LRTEASS |
| (SEQ ID NO: 2) | |
| 1 | MDCEHSLSLP CRGAEAWEIG IDKYLYAMRL SDETLIDIMT RFRKEMKNGL SRDFNPTATV |
| 61 | KMLPTFVRSI PDGSEKGDFI ALDLGGSSFR ILRVQVNHEK NQNVHMESEV YDTPENIVHG |
| 121 | SGSQLFDHVA ECLGDFMEKR KIKDKKLPVG FTFSFPCQQS KIDEAILITW TKRFKASGVE |
| 181 | GADVVKLLNK AIKKRGDYDA NIVAVVNDTV GTMMTCGYDD QHCEVGLIIG TGTNACYMEE |
| 241 | LRHIDLVEGD EGRMCINTEW GAFGDDGSLE DIRTEFDREI DRGSLNPGKQ LFEKMVSGMY |
| 301 | LGELVRLILV KMAKEGLLFE GRITPELLTR GKFNTSDVSA IEKNKEGLHN AKEILTRLGV |
| 361 | EPSDDDCVSV QHVCTIVSFR SANLVAATLG AILNRLRDNK GTPRLRTTVG VDGSLYKTHP |
| 421 | QYSRRFHKTL RRLVPDSDVR FLLSESGSGK GAAMVTAVAY RLAEQHRQIE ETLAHFHLTK |
| 481 | DMLLEVKKRM RAEMELGLRK QTHNNAVVKM LPSFVRRTPD GTENGDFLAL DLGGTNFRVL |
| 541 | LVKIRSGKKR TVEMHNKIYA IPIEIMQGTG EELFDHIVSC ISDFLDYMGI KGPRMPLGFT |
| 601 | FSFPCQQTSL DAGILITWTK GFKATDCVGH DVVTLLRDAI KRREEFDLDV VAVVNDTVGT |
| 661 | MMTCAYEEPT CEVGLIVGTG SNACYMEEMK NVEMVEGDQG QMCINMEWGA FGDNGCLDDI |
| 721 | RTHYDRLVDE YSLNAGKQRY EKMISGMYLG EIVRNILIDF TKKGFLFRGQ ISETLKTRGI |
| 781 | FETKELSQIE SDRLALLQVR AILQQLGLNS TCDDSILVKT VCGVVSRRAA QLCGAGMAAV |
| 841 | VDKIRENRGL DRLNVTVGVD GTLYKLHPHF SRIMHQTVKE LSPKCNVSFL LSEDGSGKGA |
| 901 | ALITAVGVRL RTEASS |
| (SEQ ID NO: 3) | |
| 1 | MGQICQRESA TAAEKPKLHL LAESEIDKYL YAMRLSDETL IDIMTRFRKE MKNGLSRDFN |
| 61 | PTATVKMLPT FVRSIPDGSE KGDFIALDLG GSSFRILRVQ VNHEKNQNVH MESEVYDTPE |
| 121 | NIVHGSGSQL FDHVAECLGD FMEKRKIKDK KLPVGFTFSF PCQQSKIDEA ILITWTKRFK |
| 181 | ASGVEGADVV KLLNKAIKKR GDYDANIVAV VNDTVGTMMT CGYDDQHCEV GLIIGTGTNA |
| 241 | CYMEELRHID LVEGDEGRMC INTEWGAFGD DGSLEDIRTE FDREIDRGSL NPGKQLFEKM |
| 301 | VSGMYLGELV RLILVKMAKE GLLFEGRITP ELLTRGKFNT SDVSAIEKNK EGLHNAKEIL |
| 361 | TRLGVEPSDD DCVSVQHVCT IVSFRSANLV AATLGAILNR LRDNKGTPRL RTTVGVDGSL |
| 421 | YKTHPQYSRR FHKTLRRLVP DSDVRFLLSE SGSGKGAAMV TAVAYRLAEQ HRQIEETLAH |
| 481 | FHLTKDMLLE VKKRMRAEME LGLRKQTHNN AVVKMLPSFV RRTPDGTENG DFLALDLGGT |
| 541 | NFRVLLVKIR SGKKRTVEMH NKIYAIPIEI MQGTGEELFD HIVSCISDFL DYMGIKGPRM |
| 601 | PLGFTFSFPC QQTSLDAGIL ITWTKGFKAT DCVGHDVVTL LRDAIKRREE FDLDVVAVVN |
| 661 | DTVGTMMTCA YEEPTCEVGL IVGTGSNACY MEEMKNVEMV EGDQGQMCIN MEWGAFGDNG |
| 721 | CLDDIRTHYD RLVDEYSLNA GKQRYEKMIS GMYLGEIVRN ILIDFTKKGF LFRGQISETL |
| 781 | KTRGIFETKF LSQIESDRLA LLQVRAILQQ LGLNSTCDDS ILVKTVCGVV SRRAAQLCGA |
| 841 | GMAAVVDKIR ENRGLDRLNV TVGVDGTLYK LHPHFSRIMH QTVKELSPKC NVSFLLSEDG |
| 901 | SGKGAALITA VGVRLRTEAS S |
| (SEQ ID NO: 4) | |
| 1 | MAKRALRDFI DKYLYAMRLS DETLIDIMTR FRKEMKNGLS RDFNPTATVK MLPTFVRSIP |
| 61 | DGSEKGDFIA LDLGGSSFRI LRVQVNHEKN QNVHMESEVY DTPENIVHGS GSQLFDHVAE |
| 121 | CLGDFMEKRK IKDKKLPVGF TFSFPCQQSK IDEAILITWT KRFKASGVEG ADVVKLLNKA |
| 181 | IKKRGDYDAN IVAVVNDTVG TMMTCGYDDQ HCEVGLIIGT GTNACYMEEL RHIDLVEGDE |
| 241 | GRMCINTEWG AFGDDGSLED IRTEFDREID RGSLNPGKQL FEKMVSGMYL GELVRLILVK |
| 301 | MAKEGLLFEG RITPELLTRG KFNTSDVSAI EKNKEGLHNA KEILTRLGVE PSDDDCVSVQ |
| 361 | HVCTIVSFRS ANLVAATLGA ILNRLRDNKG TPRLRTTVGV DGSLYKTHPQ YSRRFHKTLR |
| 421 | RLVPDSDVRF LLSESGSGKG AAMVTAVAYR LAEQHRQIEE TLAHFHLTKD MLLEVKKRMR |
| 481 | AEMELGLRKQ THNNAVVKML PSFVRRTPDG TENGDFLALD LGGTNFRVLL VKIRSGKKRT |
| 541 | VEMHNKIYAI PIEIMQGTGE ELFDHIVSCI SDFLDYMGIK GPRMPLGFTF SFPCQQTSLD |
| 601 | AGILITWTKG FKATDCVGHD VVTLLRDAIK RREEFDLDVV AVVNDTVGTM MTCAYEEPTC |
| 661 | EVGLIVGTGS NACYMEEMKN VEMVEGDQGQ MCINMEWGAF GDNGCLDDIR THYDRLVDEY |
| 721 | SLNAGKQRYE KMISGMYLGE IVRNILIDFT KKGFLFRGQI SETLKTRGIF ETKFLSQIES |
| 781 | DRLALLQVRA ILQQLGLNST CDDSILVKTV CGVVSRRAAQ LCGAGMAAVV DKIRENRGLD |
| 841 | RLNVTVGVDG TLYKLHPHFS RIMHQTVKEL SPKCNVSFLL SEDGSGKGAA LITAVGVRLR |
| 901 | TEASS |

As used herein, “hexokinase 2” or “hexokinase 2 isozyme” refers to any isoforms of hexokinase 2 and its naturally known variants, including that provided in SEQ ID NO: 5 as follows:

| (SEQ ID NO: 5) | |
|---|---|
| 1 | MIASHLLAYF FTELNHDQVQ KVDQYLYHMR LSDETLLEIS KRFRKEMEKG LGATTHPTAA |
| 61 | VKMLPTFVRS TPDGTEHGEF LALDLGGTNF RVLWVKVTDN GLQKVEMENQ IYAIPEDIMR |
| 121 | GSGTQLFDHI AECLANFMDK LQIKDKKLPL GFTFSFPCHQ TKLDESFLVS WTKGFKSSGV |
| 181 | EGRDVVALIR KAIQRRGDED IDIVAVVNDT VGTMMTCGYD DHNCEIGLIV GTGSNACYME |
| 241 | EMRHIDMVEG DEGRMCINME WGAFGDDGSL NDIRTEFDQE IDMGSLNPGK QLFEKMISGM |
| 301 | YMGELVRLIL VKMAKEELLF GGKLSPELLN TGRFETKDIS DIEGEKDGIR KAREVLMRLG |
| 361 | LDPTQEDCVA THRICQIVST RSASLCAATL AAVLORIKEN KGEERLRSTI GVDGSVYKKH |
| 421 | PHFAKRLHKT VRRLVPGCDV RFLRSEDGSG KGAAMVTAVA YRLADQHRAR QKTLEHLQLS |
| 481 | HDQLLEVKRR MKVEMERGLS KETHASAPVK MLPTYVCATP DGTEKGDFLA LDLGGTNFRV |
| 541 | LLVRVRNGKW GGVEMHNKIY AIPQEVMHGT GDELFDHIVQ CIADFLEYMG MKGVSLPLGF |
| 601 | TFSFPCQQNS LDESILLKWT KGFKASGCEG EDVVTLLKEA IHRREEFDLD VVAVVNDTVG |
| 661 | TMMTCGFEDP HCEVGLIVGT GSNACYMEEM RNVELVEGEE GRMCVNMEWG AFGDNGCLDD |
| 721 | FRTEFDVAVD ELSLNPGKQR FEKMISGMYL GEIVRNILID FTKRGLLFRG RISERLKTRG |
| 781 | IFETKELSQI ESDCLALLQV RAILQHLGLE STCDDSIIVK EVCTVVARRA AQLCGAGMAA |
| 841 | VVDRIRENRG LDALKVTVGV DGTLYKLHPH FAKVMHETVK DLAPKCDVSF LQSEDGSGKG |
| 901 | AALITAVACR IREAGQR |

In some examples, the 3-BP compositions described herein can further comprise a hexokinase inhibitor. The hexokinase inhibitor can be any molecule that inhibits hexokinase 1 (HK1), hexokinase 2 (HK2), and/or any isozyme thereof (collectively referred to herein as “hexokinase”).

As has been described, a major source of ATP production occurs in mitochondria in normal cells. However, ATP production from glycolysis is significantly upregulated in cancer cells. One reason for this upregulation is due to hexokinase molecules binding to, and forming a complex with, mitochondrial voltage dependent anion channels (VDACs) at ATP synthasomes, thus forming so called “ATP synthasome mega complexes.” The formation of such ATP synthasome mega complexes can immortalize the cancer cell, thus allowing the continued use of the cell's energy production processes for cancer growth. A hexokinase inhibitor, therefore, can thus block hexokinase from binding to the VADCs or displace hexokinase molecules from the VADCs of already formed ATP synthasome mega complexes.

In one example, a hexokinase inhibitor can be up to 25 amino acid units from the N-terminal region of HK1 or HK2. In another example, the hexokinase inhibitor can be an amino acid sequence of 5 to 20 amino acids, where the 5 to 20 amino acid sequence is present in the first 25 amino acid unit region of the N-terminus of HK1 or HK2. In one example, the 5 to 20 amino acid sequence can be any 5-20 amino acid sequence present in the first 25 amino acid unit region of the N-terminus of HK1 or HK2.

Such amino acid sequences can displace cellular bound hexokinase or competitively bind to voltage dependent anion channels (VDAC), thus preventing initial hexokinase binding. As has been described, a major source of ATP production occurs in mitochondria in uninfected cells. However, ATP production from glycolysis significantly increases in infected cells. Unlike normal cells, significant numbers of HK1 and/or HK2 molecules in infected cells form complexes with VDAC and ATP synthasomes to form so called “ATP synthasome mega complexes.” The formation of such ATP synthasome mega complexes can immortalize the infected cell, thus allowing the pathogen continued use of the cell's energy production processes for pathogen replication. As such, a hexokinase inhibitor can prevent or reduce the incidence of formation of ATP synthasome mega complexes, thus decreasing the pathogen's ability to replicate. Additionally, a hexokinase inhibitor can disrupt already formed ATP synthasome mega complexes, thus leading, in many cases, to apoptotic death of the cell.

In other examples, a hexokinase inhibitor can include antibodies against a portion of HK1 or HK2, such as, for example, the N-terminal region of either molecule. In one specific example, a hexokinase inhibitor can be an amino acid sequence, such as SEQ ID NO: 6, corresponding to the first 25 amino acids from the N-terminus end of hexokinase 1 (isoform1) having a sequence as follows:

| (SEQ ID NO: 6) | |
|---|---|
| 1 | MIAAQLLAYY FTELKDDQVK KIDKY |

In another example, a hexokinase inhibitor can be an amino acid sequence as in SEQ ID NO: 7, corresponding to the first 25 amino acids from the N-terminus end of hexokinase 1 (isoform 2) having a sequence as follows:

| (SEQ ID NO: 7) | |
|---|---|
| 1 | MDCEHSLSLP CRGAEAWEIG IDKYL |

In yet another example, a hexokinase inhibitor can be an amino acid sequence as in SEQ ID NO: 8, corresponding to the first 25 amino acids from the N-terminus end of hexokinase 1 (isoform 3) having a sequence as follows:

| (SEQ ID NO: 8) | |
|---|---|
| 1 | MGQICQRESA TAAEKPKLHL LAESE |

In still another example, a hexokinase inhibitor can be an amino acid sequence as in SEQ ID NO: 9, corresponding to the first 25 amino acids from the N-terminus end of hexokinase 1 (isoform 4) having a sequence as follows:

| (SEQ ID NO: 9) | |
|---|---|
| 1 | MAKRALRDFI DKYLYAMRLS DETLI |

In yet another example, a hexokinase inhibitor can be an amino acid sequence as in SEQ ID NO: 10, corresponding to the first 25 amino acids from the N-terminus end of hexokinase 2 having a sequence as follows:

(SEQ ID NO: 10)
MIASHLLAYF FTELNHDQVQ KVDQY

Additional hexokinase inhibitors can be those as disclosed in U.S. Pat. No. 5,854,067 (to Newgard et al, issued Dec. 29, 1998) and/or U.S. Pat. No. 5,891,717 (to Newgard et al., issued Apr. 6, 1999), both of which are incorporated by reference in their entireties. Additional hexokinase inhibitors that can be used in the present formulations include those disclosed in U.S. Pat. Nos. 6,670,330; 6,218,435; 5,824,665; 5,652,273; and 5,643,883; and U.S. patent application publication Nos. 20030072814; 20020077300; and 20020035071; each of the foregoing patent publications and patent application is incorporated herein by reference, in their entireties.

In some examples, the present 3-BP compositions can further include various additives. In one example, a composition can include immune system modulators and/or immune system boosters. Such immune system modulators and/or immune system boosters can include, without limitation, d-lactic acid, epinephrine, brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, antibodies against nagalase, etc. Without being bound by any particular theory, flavonoids may have indirect immune modulating effects. Specifically, increase in antioxidant capacity of blood seen after the consumption of flavonoid-rich foods may not caused directly by flavonoids themselves, but may be due to increased uric acid levels that result from metabolism of flavonoids. The body sees them as foreign compounds and is trying to get rid of them. This process of removing unwanted compounds includes Phase II enzymes that also help eliminate mutagens and carcinogens, and therefore may be of value. In one embodiment, the present compositions can include d-lactic acid. In another embodiment, the present compositions can include epinephrine.

In some examples, the present 3-BP compositions can comprise antifungal agents, antibiotics, glycolysis inhibitors, inhibitors of mitochondria, sugars, and biological buffers, without limitation. Examples of such agents include, but are not limited to, amphotericin B, efrapeptin, doxorubicin, 2-deoxyglucose (2DOG), d-lactic acid, analogs of 2DOG, dicholoracetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. Various active agents of the composition can include a cellular energy inhibitor, a glycolysis inhibitor, a mitochondria inhibitor, a halo monocarboxylate compound, an antifungal agent, an antibiotic agent, and the like.

In one embodiment, the present compositions can include phospholipids including liposomes and nanoparticles. The liposomes or nano-particles can incorporate annexin-A5 molecules or antibodies against phosphatidylserine. In this way, the rate of 3BP release can be controlled and its delivery can be targeted. In other examples, the present compositions can include L-Lactate dehydrogenase, D-Lactate Dehydrogenase, or both. In other examples, the present compositions can include nicotinamide adenine dinucleotides (NAD⁺), which can be included in the present formulations to decrease the blood lactate concentration as well as the lactate concentration near infected cells. By decreasing the blood lactate concentration in infected subjects, the highly glycolytic innate immune system can work appropriately.

In one embodiment, the present compositions can include less biologically active amino acids as compared to their isomers to facilitate infected cell starvation. In one aspect, the less biologically active amino acid can be a D-amino acid. However, if the L-amino acid is less biologically active than the D-form, the L-amino acid can be used.

In one embodiment, the present compositions can include inhibitors for DNA replication: inhibitors for DNA binding; and/or inhibitors for DNA transcription. In another embodiment, the present compositions can include inhibitors for cell cycle, growth and/or proliferation. In yet another embodiment, the present compositions can include inhibitors for signal transduction pathways. In yet another embodiment, the present compositions can include inhibitors for angiogenesis. In yet another embodiment, the present compositions can include small RNAs that interfere with normal gene control including antisense RNA, micro RNA, small hairpin RNA, short hairpin RNA, small interfering RNA, and the like. In yet another embodiment, the present compositions can include vitamin C: nutritional supplements including vitamins, CoQ10, flavonoids, free fatty acid, alpha lipoic acid, acai, gogi, mango, pomergrante, L-carnitine, selenium; etc.

In addition to the active agent(s), the composition can also include a pharmaceutically acceptable carrier. The carrier can be a single composition, or a mixture of compositions. Additionally, the carrier can take the form of an encapsulation coat, an absorbing agent, a coating substance, a controlled release device, a release modifying agent, surfactants, or a combination thereof. In some aspects, the carrier can comprise about 1 wt % to about 99 wt % of the total composition. In one embodiment, the carrier can comprise about 5 wt % to about 95 wt % of the total formulation. In another embodiment, the carrier can comprise about 20 w1% to about 80 wt %. In yet a further embodiment, the carrier can comprise about 30 wt % to about 60) w1%. In one embodiment, the carrier can be admixed with the active agent(s). In another embodiment, the carrier can adsorb, entrap, or encapsulate at least a portion of the active agent(s).

Non-limiting examples of compounds that can be used as at least a part of the carrier include without limitation; cetyl alcohol and its esters; stearic acid and its glycerol esters, polyoxyethylene alkyl ethers; polyethylene glycol; polyglycolyzed glycerides; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; proteins; polyoxyethylene glycerides; polyoxyethylene sterols, derivatives, and analogues thereof; polyoxyethylene hydrogenated vegetable oils; reaction mixtures of polyols with at least one member of the group consisting of fatty acids, glycerides, vegetable oils, hydrogenated vegetable oils, and sterols: tocopherol derivatives, sugar esters: sugar ethers: sucroglycerides: waxes, shellac, pharmaceutically acceptable salts thereof, and mixtures thereof.

Non-limiting examples of release modifying agents include without limitation: polyethylene glycols having a weight average molecular weight of about 1000 and more, carbomer, methyl methacrylate copolymers, methacrylate copolymers, hydroxypropyl methyl cellulose, hydroxypropyl cellulose, cellulose acetate phthalate, ethyl cellulose, methyl cellulose and their derivatives: ion-exchange resin: mono-, di-, tri-esters of fatty acids with glycerol: tocopherol and its esters: sucrose esters with fatty acids: polyvinyl pyrollidone: xanthan gums: cetyl alcohol: waxes: fats and oils, proteins, alginate, polyvinyl polymers, gelatins, organic acids, and their derivatives and combinations thereof.

In one embodiment, the carrier can include at least one of celluloses; carbomers; methacrylates; dextrins; gums; inorganic carbonates or salts of calcium or magnesium or both; fatty acid esters; gelatin; lactoses; maltoses; mono-, di- or triglycerides; oils; polyethylene glycols; polyethylene oxide co-polymers; proteins; resins; shellac; silicates; starches; sugar stearates; partially or fully hydrogenated vegetable oils; waxes; and combinations thereof.

In yet another embodiment, the carrier can include at least one of celluloses; carbomers; methacrylates; inorganic carbonates or salts of calcium; inorganic carbonates or salts of magnesium; fatty acids; fatty acid esters; gelatin; lactoses; polyethylene glycol; polyethylene oxide co-polymers; silicates; partially or fully hydrogenated vegetable oils, and combinations thereof.

In yet a further embodiment, the carrier can include at least one of microcrystalline cellulose: hydroxypropyl methylcellulose; ethyl cellulose: silicon dioxide: magnesium aluminosilicate: lactose: xanthan gum: stearic acid: glyceryl distearate: hydrogenated vegetable oil; and combinations thereof.

The formulation, including any dosage form, can include other components or additives. Such additional components and additives are optional. In one aspect, the additive can be a solid at room temperature and have a melting point or range that is greater than about 40° C. Non-limiting examples of additives that can be included in the systems of the present invention include without limitation: fillers such as lactoses, starches, sugars, celluloses, calcium salts, silicon oxides, metallosilicates and the like; disintegrants such as starch glycolate, lauryl sulfate, pregaltinized starch, croscarmellose, crospovidone and the like; binders such as pyrrolidones, methacrylates, vinyl acetates, gums, acacia; tragacanth; kaolins; carrageenan alginates, gelatins and the like; cosolvents such as alcohols, polyethylene glycols having average molecular weight of less than 1000, propylene glycols and the like; surface tension modifiers such as hydrophilic or amphiphlic surfactants; taste-masking agents; sweeteners; microencapsulating agents; process aids such as lubricants, glidants, talc, stearates, lecithin and the like; polymeric coating agents; plasticizers; buffers; organic acids; antioxidants; flavors; colors; alkalizers; humectants; sorbitols; mannitols; osmotic salts; proteins; resins; moisture repelling agents; hygroscopic agents; desiccants; and combinations thereof.

The formulations of the present invention can be formulated into a variety of oral dosage forms including, but not limited to two piece hard gelatin capsules, soft gelatin capsules, beads, beadlets, granules, spherules, pellets, microcapsules, microspheres, nanospheres, nanocapsules, tablets, or combinations thereof. Other forms known to those of ordinary skill in the art may also be used. In one aspect, the oral dosage form may be a capsule or tablet. In another embodiment the oral dosage form may include a multi-component dosage form such as beads in a capsule, a capsule or capsules within a capsule, a tablet or tablets in a capsule, or a multilayer tablet. It is noteworthy that, when the formulation includes multiple dosage forms, such dosage forms need not be the same. Further, such dosage forms may not be physically present together.

The dosage form, e.g. tablet, may be coated or enrobed with a hydrophilic or a hydrophobic coat material known in the art. In one embodiment, the coat can be a film coat, sugar coat, enteric coat, semipermeable coat, sustained release coat, delayed release coat, osmotic coat and the like. In a further embodiment, the coating material can be a cellulose, gelatin, methacrylate, polyvinyl acetate, povidone, polyethylene glycol, polyetyhylne oxide, poloxamers, carbomers, shellac, phthalate and the like and their derivatives and combinations thereof. In another embodiment, the coat is a dry powder coat. In one embodiment, the tablet can be a matrix tablet. It is noteworthy that, when present, the coat can be considered as part, or all, of the carrier component of the formulation.

In addition to the compositions described herein, a method for the treatment of a pathogenic infection can comprise administering to a subject a 3-BP composition as described herein in a therapeutically effective amount. In one example, the composition can be administered to the subject when the subject's blood insulin/glucagon ratio is in the range of about 1 to about 10. In another example, the composition can be administered to the subject after fasting for at least 4 hours. In yet another example, the composition can be administered to the subject after fasting for 6 hours, and in another embodiment, after fasting for 8 hours. In another example, the composition can be administered to the subject after fasting for 2 hours. It is noted that such times are not intended to be limiting, and that in one embodiment, the amount of time fasting can be such that the subject's blood insulin/glucagon ratio is in the range of about 2 to about 5.

### EXAMPLES

In one example, a composition for protecting a subject against, or treating a subject with, a pathogenic infection can include a cellular energy inhibitor having the structure according to formula I

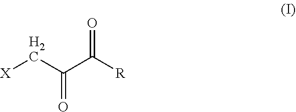

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal: where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl;
at least one sugar, which stabilizes the cellular energy inhibitor by substantially preventing the inhibitor from hydrolyzing; and
a biological buffer that is present in an amount sufficient to at least partially deacidify the cellular energy inhibitor and neutralize metabolic by-products of the cellular energy inhibitor.

In one example, the cellular energy inhibitor is a 3-halopyruvate selected from 3-fluoropyruvate, 3-chloropyruvate, 3-bromopyruvate, 3-iodopyruvate, and combinations thereof.

In one example, the cellular energy inhibitor is 3-bromopyruvate.

In one example, the at least one sugar can be selected from gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, or a combination thereof.

In one example, the at least one sugar can be a five-carbon sugar.

In one example, the at least one sugar can be at least two five-carbon sugars.

In one example, the composition can include a second sugar selected from mannitol, erytritol, isomalt, lactitol, maltitol, sorbitol, xyolitol, dulcitol, ribitol, inositol, sorbitol, and combinations thereof.

In one example, the composition can include a second sugar and a third sugar independently selected from mannitol, erytritol, isomalt, lactitol, maltitol, sorbitol, xyolitol, dulcitol, ribitol, inositol, sorbitol, and combinations thereof.

In one example, the at least one sugar can include glycerol, inositol, and sorbitol.

In one example, the composition includes glycerol in a range from about 0.1 wt % to about 3 wt %, inositol in a range from about 1 wt % to about 5 wt %, and sorbitol in a range from about 30 wt % to about 50 wt %.

In one example, the composition can include d-lactic acid and epinephrine.

In one example, the composition can include a glycolysis inhibitor and wherein the glycolysis inhibitor is 2-deoxglucose in a concentration from about 1 mM to about 5 mM.

In one example, the composition can include the glycolysis inhibitor 2-deoxglucose.

In one example, the composition can include the 2-deoxglucose in a concentration from about 1 mM to about 5 mM.

In one example, the biological buffer is selected from a citrate buffer, a phosphate buffer, and an acetate buffer.

In one example, the biological buffer is a citrate buffer.

In one example, the composition can include at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof. In one example, the composition can include a hexokinase inhibitor.

In one example, the hexokinase inhibitor inhibits binding of hexokinase 1 and/or hexokinase 2 to VDAC.

In one example, the hexokinase inhibitor is an amino acid sequence selected from the group consisting of: SEQ ID NO: 6, SEQ ID NO: 7, SEQ ID NO: 8, SEQ ID NO: 9, and SEQ ID NO. 10.

In one example, the composition can include an antifungal agent and/or antibacterial agent.

In one example, the composition can include an antifungal agent and/or antibacterial agent in a concentration from about 0.05 mM to about 0.5 mM.

In one example, the composition can include a mitochondrial inhibitor.

In one example, the mitochondrial inhibitor is selected from oligomycin, efrapeptin, aurovertin, and mixtures thereof; in a concentration from about 0.01 mM to about 0.5 mM.

In one example, the mitochondrial inhibitor is in a concentration from about 0.01 mM to about 0.5 mM.

In another example, a method for protecting a subject against, or treating a subject with, a pathogenic infection can include administering to a subject the composition of claim 1 in a therapeutically effective amount.

In another example, the composition is administered to the subject enterally, parenterally, transdermally, or by a combination thereof.

In another example, a composition for treating or preventing a secondary infection in a subject having a primary pathogenic infection, can include a cellular energy inhibitor having a structure according to formula I

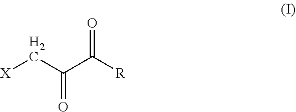

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal: where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl. The composition can also include at least one sugar that stabilizes the cellular energy inhibitor by substantially preventing the inhibitor from hydrolyzing and a biological buffer present in an amount sufficient to at least partially deacidify the cellular energy inhibitor and neutralize metabolic by-products of the cellular energy inhibitor.

In one example, the secondary infection can include a coinfection, a superinfection, an opportunistic infection, or a combination thereof.

In another example, a method for treating or preventing secondary infections in a subject having, or having recently had, a pathogenic primary infection, can include administering a halopyruvate composition to the subject, the halopyruvate composition including a cellular energy inhibitor having a structure according to formula I

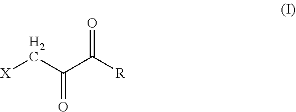

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal: where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl. The halopyruvate composition also includes at least one sugar that stabilizes the cellular energy inhibitor by substantially preventing the inhibitor from hydrolyzing and a biological buffer present in an amount sufficient to at least partially deacidify the cellular energy inhibitor and neutralize metabolic by-products of the cellular energy inhibitor.

In one example, the halopyruvate composition is administered concurrently with an active agent

In yet another example, composition for reducing or preventing hyper-glycolysis in leukocytes of a subject having a primary pathogenic infection can include a cellular energy inhibitor having a structure according to formula I

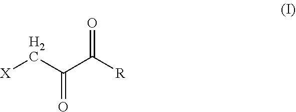

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal: where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl. The example additionally includes at least one sugar that stabilizes the cellular energy inhibitor by substantially preventing the inhibitor from hydrolyzing and a biological buffer present in an amount sufficient to at least partially deacidify the cellular energy inhibitor and neutralize metabolic by-products of the cellular energy inhibitor.

In yet another example, a method for reducing or preventing hypercytokinemia in a subject having a primary pathogenic infection can include administering a halopyruvate composition to the subject, the halopyruvate composition including a cellular energy inhibitor having a structure according to formula I

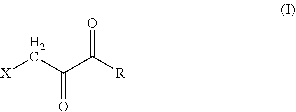

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal; where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl. The composition can further include at least one sugar that stabilizes the cellular energy inhibitor by substantially preventing the inhibitor from hydrolyzing and a biological buffer present in an amount sufficient to at least partially deacidify the cellular energy inhibitor and neutralize metabolic by-products of the cellular energy inhibitor.

In yet another example, a composition for reducing hyper-glycolysis in hyperactivated non-leukocyte cells of a subject having a pathogenic infection can include a cellular energy inhibitor having a structure according to formula I

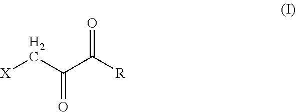

wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal; where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl. The composition can additionally include at least one sugar that stabilizes the cellular energy inhibitor by substantially preventing the inhibitor from hydrolyzing and a biological buffer present in an amount sufficient to at least partially deacidify the cellular energy inhibitor and neutralize metabolic by-products of the cellular energy inhibitor.

What is claimed is:

1. A method for protecting a subject against, or treating a subject with, a pathogenic infection, comprising:
administering to a subject a therapeutically effective amount of a composition including a cellular energy inhibitor having the structure according to formula I 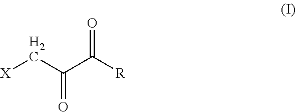
wherein X is selected from the group consisting of: a nitro, an imidazole, a halide, sulfonate, a carboxylate, an alkoxide, and amine oxide; and R is selected from the group consisting of: OR′, N(R″)₂, C(O)R′″, C1-C6 alkyl, C6-C12 aryl, C1-C6 heteroalkyl, a C6-C12 heteroaryl, H, and an alkali metal; where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl;
at least one sugar, which stabilizes the cellular energy inhibitor by substantially preventing the inhibitor from hydrolyzing; and
a biological buffer that is present in an amount sufficient to at least partially deacidify the cellular energy inhibitor and neutralize metabolic by-products of the cellular energy inhibitor.

2. The method of claim 1, wherein the cellular energy inhibitor is 3-bromopyruvate.

3. The method of claim 1, wherein the at least one sugar is a member selected from the group consisting of gluconic acid, glucuronic acid, mannitol, erythritol, isomalt, lactitol, maltitol, sorbitol, xylitol, dulcitol, ribitol, inositol, glycerol, ethylene glycol, threitol, arabitol, galactitol, fucitol, iditol, volemitol, maltotriitol, maltotetraitol, polyglycitol, and a combination thereof.

4. The method of claim 1, further comprising a second sugar selected from the group consisting of mannitol, erythritol, isomalt, lactitol, maltitol, sorbitol, xylitol, dulcitol, ribitol, inositol, sorbitol, and combinations thereof.

5. The method of claim 1, wherein the composition can include a second sugar and a third sugar independently selected from mannitol, erytritol, isomalt, lactitol, maltitol, sorbitol, xyolitol, dulcitol, ribitol, inositol, sorbitol, or a combination thereof.

6. The method of claim 1, the composition further comprising at least one sugar selected from glycerol, inositol, and sorbitol.

7. The method of claim 1, the composition further comprising d-lactic acid and epinephrine.

8. The method of claim 1, further comprising a glycolysis inhibitor.

9. The method of claim 8, wherein the glycolysis inhibitor is 2-deoxglucose.

10. The method of claim 9, wherein the 2-deoxglucose is in a concentration from about 1 mM to about 5 mM.

11. The method of claim 1, wherein the biological buffer is selected from a citrate buffer, a phosphate buffer, and an acetate buffer.

12. The method of claim 1, wherein the biological buffer is a citrate buffer.

13. The method of claim 1, the composition further comprising at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof.

14. The method of claim 1, the composition further comprising a hexokinase inhibitor.

15. The method of claim 14, wherein the hexokinase inhibitor inhibits binding of hexokinase 1 and/or hexokinase 2 to VDAC.

16. The method of claim 1, wherein the hexokinase inhibitor is an amino acid sequence selected from the group consisting of: SEQ ID NO: 6, SEQ ID NO: 7, SEQ ID NO: 8, SEQ ID NO: 9, and SEQ ID NO. 10.
